(19) United States
(12) Patent Application Publication
(10) Pub. No.: US 2022/0196833 A1
(43) Pub. Date: Jun. 23, 2022

(54) Method for Determining a Geometry of an Ear Canal or a Portion of an Ear of a Person

(71) Applicant: SONOVA AG, Staefa (CH)

(72) Inventor: Erwin Kuipers, Staefa (CH)

(21) Appl. No.: 17/536,504

(22) Filed: Nov. 29, 2021

(30) Foreign Application Priority Data
Dec. 23, 2020 (EP) EP20216877.9

(51) Int. Cl. G01S 15/89; G01S 7/52

(52) U.S. Cl. CPC G01S 15/899; G01S 7/52025; G01S 7/5202; G01S 15/8915

(57) ABSTRACT

A method for determining a geometry of an ear canal or a portion of an ear of a person may include filling the ear canal and/or the portion of the ear with a liquid or a gel, inserting a capacitive micromachined ultrasonic transducer probe or a piezoelectric micromachined ultrasonic transducer probe, acquiring data using the probe, and processing the acquired data to obtain a 2D or 3D image of the ear canal and/or the portion of the ear.

### RELATED APPLICATIONS

The present application claims priority to EP Patent Application No. EP 20216877.9, filed Dec. 23, 2020, the contents of which are hereby incorporated by reference in their entirety.

### BACKGROUND INFORMATION

When providing hearing impaired people with hearing aids it is desirable to use comfortable earpieces with adequate retention. This requires detailed knowledge of the ear canal and outer ear geometry. In addition, the location of the osseous/cartilaginous (also referred to as bony/cartilaginous junction) should be known. However, to identify the spatial zones in which overwaxing can be applied, knowledge of the local radial ear canal tissue compliance is desired.

Overwaxing is a term used for locally offsetting the earpiece wall radially, mostly for obtaining an acceptable wearing comfort and retention. Another reason for performing a certain level of overwaxing is to obtain a more tight acoustic sealing to enhance the hearing performance.

### BRIEF DESCRIPTION OF THE DRAWINGS

The present disclosure will become more fully understood from the detailed description given hereinbelow and the accompanying drawings which are given by way of illustration only, and thus, are not limitive of the present disclosure, and wherein:

FIG. 1 is a schematic view illustrating an exemplary method for determining a geometry of an ear canal or a portion of the ear of a person.

### DETAILED DESCRIPTION

Described herein is a method for determining a geometry of an ear canal or a portion of an ear of a person.

It is a feature of the present disclosure to provide an improved method for determining a geometry of an ear canal or a portion of an ear of a person.

In some examples, a method for determining a geometry of an ear canal or a portion of an ear, e.g. a portion of an outer ear, of a person may include filling the ear canal and/or the portion of the ear with a liquid or a gel, inserting a capacitive micromachined ultrasonic transducer (CMUT) probe or a piezoelectric micromachined ultrasonic transducer (PMUT), acquiring data using the probe, and processing the acquired data to obtain a 2D or 3D image of the ear canal and/or the portion of the ear. The measurement using a capacitive micromachined ultrasonic transducer probe is very fast (e.g. up to 40/100 MHz ultrasound frequency, the sampling rate can be very high). The measurement can be performed when the probe has been inserted and is located at an insertion depth or it can be performed also during the insertion process, while the probe is being moved. The patient will never be in perfect rest. Tissue movement due to varying blood flow strength and body movement due to respiration are some causes for this. It will be possible to determine the ear canal geometry based on ultrasound measurements using a correlation technique, even if the probe is in movement.

In an exemplary embodiment the ear canal is cleaned and/or inspected prior to filling in the liquid or gel.

In an exemplary embodiment the liquid or gel is tempered to a temperature of 15° C. to 50° C., in particular 28° C. to 38° C., especially 28° C. to 35° C., before being filled in.

In an exemplary embodiment, the person is positioned lying sideways before filling in the liquid or gel.

In an exemplary embodiment, during insertion, an actual insertion depth is monitored. This may be done by the probe or by one or more additional sensors.

In an exemplary embodiment, a warning is output when the probe reaches or falls below a predetermined distance to an eardrum of the ear. The design of the probe may be such, that touching the ear canal wall would not injure the skin. A soft material could be used as a bumper.

In an exemplary embodiment, at the desired insertion depth, the probe is fixated in position and/or orientation by at least one of a headband, a strap and a skin adhesive.

In an exemplary embodiment, processing the acquired data further includes determining mechanical properties of the wall of the ear canal and/or the underlying tissue. For example, mechanical properties may be determined by sending out ultrasonic waves and analyzing the reflected waves at different time instants. By changing the angle of the emitted waves, or by focusing the ultrasound on a point in the tissue, 3D spatial information can be obtained. Signal processing techniques like averaging, filtering, transform to the frequency domain may be applied. The mechanical properties may include visco-elastic properties, isotropic/anisotropic properties of the tissue and visco-plastic properties. Moreover, the mechanical properties determined may relate to a visco-elastic model.

In an exemplary embodiment, processing the acquired data further includes performing 3D mapping of osseous material surrounding the ear canal and determining a location of an osseous/cartilaginous junction. The change from tissue to osseous material is such that impinging ultrasonic waves will be reflected at the transition tissue-osseous material. By employing imaging techniques like array-based beamforming and -focusing, a 3D map can be generated by signal processing.

In an exemplary embodiment, processing the acquired data further includes performing a 3D elastography, generating a 3D map of elastic moduli of tissue surrounding the ear canal.

In an exemplary embodiment, processing the acquired data further includes performing a 3D mapping of at least one biometric parameter.

In an exemplary embodiment, the at least one biometric parameter is one of blood perfusion and fat content.

In an exemplary embodiment, processing the acquired data includes determining information about the length of the ear canal.

In an exemplary embodiment, the acquired and/or processed data are stored in a cloud. A cloud is understood to be a remote file hosting service designed to host user files.

According to an aspect of the present disclosure, a measurement device is provided, comprising a capacitive micromachined ultrasonic transducer probe or a piezoelectric micromachined ultrasonic transducer and a data processing unit configured to perform the data acquisition and processing according to the method as described above.

Employing the proposed method, multiple purposes can be served:
3D Scanning of the ear canal shape,
Determination of the length and/or volume of the ear canal,
Determination of the location of the bony/cartilaginous junction,
Determination of mechanical tissue properties,
Identification of outer ear diseases,
Identification of general health related and biometric parameters.

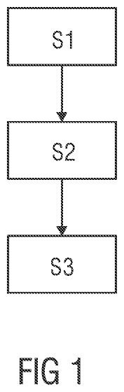

FIG. 1 is a schematic view illustrating an exemplary method for determining a geometry of an ear canal or a portion of ear, in particular a portion of an outer ear, of a person.

The method for determining a geometry of an ear canal or a portion of the ear of a person procedure is proposed as follows:

An ear canal and outer ears of a person may be inspected according to common audiological working standards, including check for infections, cerumen, diseases, etc. If necessary, the ear canal and/or outer ear may be cleaned.

In a step S1, the ear canal and/or the portion of the ear, in particular the outer ear, be filled with a liquid or a gel for improving ultrasound transmission. The user may be positioned lying sideways for this purpose. In an exemplary embodiment, the liquid or gel is tempered to a temperature of 15° C. to 50° C., in particular 28° C. to 38° C., preferably 28° C. to 35° C. before being filled in.

In a step S2, a capacitive micromachined ultrasonic transducer probe or a piezoelectric micromachined ultrasonic transducer probe is inserted into the ear canal. During insertion, an actual insertion depth may be monitored, e.g. by the probe and/or using one or more additional sensors. A warning may be output when the probe reaches or falls below a predetermined distance to an eardrum of the ear. When the probe has reached the desired insertion depth, the probe may be fixated in position and/or orientation by at least one of a headband, a strap and a skin adhesive.

Once inserted and/or during insertion data may be acquired by the probe in a step S3, wherein the acquired data are processed to obtain a 2D or 3D image of the ear canal and/or the portion of the ear.

Processing data may further include one or more of:
determining mechanical properties of the wall of the ear canal or the underlying tissue,

performing 3D mapping of osseous material surrounding the ear canal and determining a location of an osseous/cartilaginous junction,

performing a 3D elastography, generating a 3D map of elastic moduli of tissue surrounding the ear canal,

performing a 3D mapping of at least one biometric parameter such as blood perfusion and fat content,

determining information about the length and/or volume of the ear canal,

performing a 3D mapping of the eardrum.

Mechanical properties may for example comprise the elastic modulus of the tissue, the tensile strength or hardness of the tissue. In order to determine such properties the invention proposes the use of a CMUT or PMUT. The ear canal, or the part of the ear under investigation is exposed to an ultrasound signal or a pulse signal. This signal travels to the wall of the ear canal and exerts a mechanical force on the surface and the underlying tissue. The reflected signal is detected by the CMUT or PMUT which acts as a hydrophone array. In medical imaging CMUTs have been used for examination of blood vessels. Since the ear canal is normally not filled with a liquid or gel CMUTs have not been used for this purpose. By measuring the time-delay between the ultrasound or pulse signal and the signal as picked up by the CMUT array the geometry of the ear canal can be determined. The time delay between the generation of the pulse and the arrival of the response in the single transducers of the CMUT array is proportional to the length the pulse has traveled through the liquid or gel. This allows reconstructing an image of the ear canal.

A reflected signal is dampened if the reflected surface is soft. From the intensity of the reflected signal the softness or dampening characteristic can be derived by a technician who is skilled in the art.

After the data acquisition the probe may be removed from the ear canal, the liquid or gel may be removed and the process may be repeated for the other ear of the person, if required.

The acquired and/or processed data may be stored in a secure way, e.g. they may be anonymized and stored in a cloud for cloud-based statistical analysis.

In an exemplary embodiment, processing of data may include forming a 3D elastic map (Young's modulus, shear modulus) by combining multiple slices.

In an exemplary embodiment the probe may be made of a more or less flexible material, where the transducer arrays are placed on a rigid section of the probe. For example, the probe may comprise a flexible printed circuit board or a flexible substrate which allows for folding or wrapping processes without damaging the electrical network.

In an exemplary embodiment the probe has a diameter of at most 6 millimeters.

In an exemplary embodiment the probe may have an elliptic or circular cross-sectional shape.

In an exemplary embodiment the probe has a smooth outer surface to not harm the patient.

In an exemplary embodiment, the probe may be equipped with a transducer array at its medial end to facilitate monitoring the distance to the eardrum.

In an exemplary embodiment, the probe may comprise an optical waveguide or a miniature camera for visual inspection purposes during the insertion process.

S1, S2, S3 refer to steps herein.

The method may be employed for ear canal and tissue imaging in order to allow for an optimal fitting and -wearing comfort of an earpiece, e.g. for a hearing device such as a hearing aid or an ear bud or any other type of hearing instrument.

What is claimed is:

1. A method for determining a geometry of one of an ear canal and a portion of an ear of a person, the method comprising:
filling at least one of the ear canal and the portion of the ear with one of a liquid and a gel,
inserting one of a capacitive micromachined ultrasonic transducer probe and a piezoelectric micromachined ultrasonic transducer probe,
acquiring data using the probe,
processing the acquired data to obtain one of a 2D and a 3D image of at least one of the ear canal and the portion of the ear.

2. The method according to claim 1, wherein the data are acquired when the micromachined ultrasonic transducer probe has been inserted and is located at an insertion depth.

3. The method according to claim 1, wherein the data are acquired during the insertion process while the micromachined ultrasonic transducer probe is being moved.

4. The method of claim 1, wherein the ear canal is at least one of cleaned and inspected prior to filling in the one of the liquid and the gel.

5. The method according to claim 1, wherein the one of the liquid and the gel is tempered to a temperature of 15° C. to 50° C. before being filled in.

6. The method according to claim 1, wherein the one of the liquid and the gel is tempered to a temperature of 28° C. to 38° C. before being filled in.

7. The method according to claim 1, wherein the person is positioned lying sideways.

8. The method according to claim 1, wherein during insertion, an actual insertion depth is monitored.

9. The method of claim 8, wherein a warning is output when the probe reaches a predetermined distance to an eardrum of the ear.

10. The method of claim 8, wherein a warning is output when the probe falls below a predetermined distance to an eardrum of the ear.

11. The method according to claim 1, wherein at the desired insertion depth, the probe is fixated in at least one of position and orientation by at least one of a headband, a strap and a skin adhesive.

12. The method according to claim 1, wherein processing the acquired data further includes determining mechanical properties of at least one of the wall of the ear canal and the underlying tissue.

13. The method according to claim 1, wherein processing the acquired data further includes performing 3D mapping of osseous material surrounding the ear canal and determining a location of an osseous/cartilaginous junction.

14. The method according to claim 1, wherein processing the acquired data further includes performing a 3D elastography, generating a 3D map of elastic moduli of tissue surrounding the ear canal.

15. The method according to claim 14, wherein the at least one biometric parameter is one of blood perfusion and fat content.

16. The method according to claim 1, wherein processing the acquired data further includes performing a 3D mapping of at least one biometric parameter.

17. The method according to claim 1, wherein processing the acquired data includes at least one of determining information about at least one of the length and volume of the ear canal and performing a 3D mapping of an eardrum.

18. The method according to claim 1, wherein at least one of the acquired and processed data are stored in a cloud.

19. A measurement device comprising one of a capacitive micromachined ultrasonic transducer probe and a piezoelectric micromachined ultrasonic transducer probe, and a data processing unit configured to determine a geometry of one of an ear canal and a portion of an ear of a person, by:
filling at least one of the ear canal and the portion of the ear with one of a liquid and a gel,
inserting the one of the capacitive micromachined ultrasonic transducer probe and the piezoelectric micromachined ultrasonic transducer probe,
acquiring data using the probe,
processing the acquired data to obtain one of a 2D and a 3D image of at least one of the ear canal and the portion of the ear.
